(19) United States
(12) Patent Application Publication
(10) Pub. No.: US 2025/0285270 A1
(43) Pub. Date: Sep. 11, 2025

(54) VIRTUAL STAINING BASED ON MULTIPLE SETS OF IMAGING DATA HAVING MULTIPLE PHASE CONTRASTS

(71) Applicant: Carl Zeiss Microscopy GmbH, Jena (DE)

(72) Inventors: Lars LOETGERING, Jena (DE); Marika VALENTINO, Santa Maria a Vico (CE) (IT)

(73) Assignee: Carl Zeiss Microscopy GmbH, Jena (DE)

(21) Appl. No.: 19/072,433

(22) Filed: Mar. 6, 2025

(30) Foreign Application Priority Data
Mar. 7, 2024 (DE) 102024106574.5

(51) Int. Cl. G06T 7/00; G01N 1/30; G06T 5/50; G16H 30/20; G16H 30/40

(52) U.S. Cl. CPC G06T 7/0012; G01N 1/30; G06T 5/50; G16H 30/20; G16H 30/40; G06T 2207/20221; G06T 2207/30024

(57) ABSTRACT

Various examples of the disclosure are directed to techniques of virtually staining a tissue sample. One or more output images having a virtual stain are determined. The techniques aim at a more robust process. For this, multiple sets of imaging data serve as an input to the digital process, the multiple sets of imaging data having multiple different phase contrasts.

### FIELD OF THE INVENTION

Various embodiments relate to techniques for virtual staining by utilizing a machine-learning logic. Various examples specifically relate to processing multiple sets of imaging data having different phase contrasts.

### BACKGROUND OF THE INVENTION

Histopathology is an important tool in the diagnosis of a disease. Histopathology refers to the optical examination of tissue samples. Diagnosis of cells in the tissue sample is facilitated.

Typically, histopathological examination starts with surgery, biopsy, or autopsy for obtaining the tissue to be examined. The tissue may be processed to remove water and to prevent decay. The processed sample may then be embedded in a wax block. From the wax block, thin sections may be cut. Said thin sections may be referred to as tissue samples hereinafter.

The tissue samples may be analyzed by a histopathologist in a microscope. The tissue samples may be stained with a chemical stain using an appropriate staining laboratory process, to thereby facilitate the analysis of the tissue sample. In particular, chemical stains may reveal cellular components which are very difficult to observe in the unstained tissue sample. Moreover, chemical stains may provide contrast. The chemical stains may highlight one or more biomarkers or predefined structures of the tissue sample.

The most used chemical stain in histopathology is a combination of haematoxylin and eosin (abbreviated H&E). Haematoxylin is used to stain nuclei blue, while eosin stains cytoplasm and the extracellular connective tissue matrix pink. There are hundreds of various other techniques which have been used to selectively stain cells. Recently, antibodies have been used to stain particular proteins, lipids and carbohydrates. Called immunohistochemistry, this technique has greatly increased the ability to specifically identify categories of cells under a microscope. Staining with an H&E stain may be considered as common gold standard for histopathologic diagnosis.

By coloring tissue samples with chemical stains, otherwise almost transparent and indistinguishable structures/tissue sections of the tissue samples become visible for the human eye. This allows pathologists and researchers to investigate the tissue sample under a microscope or with a digital bright-field equivalent image and assess the tissue morphology (structure) or to look for the presence or prevalence of specific cell types, structures or even microorganisms such as bacteria.

Preferably, several chemical stains are used to fully assess the pathology case. Typically, only one chemical stain can be applied to a tissue sample. Thus, if several chemical stains are required for diagnosis, several tissue samples have to be prepared. Moreover, different chemical stains may require different staining protocols. Thus, the known chemical staining techniques are labour- and cost-intensive.

WO 2019/154987 A1 discloses a method providing a virtually stained image looking like a typical image of a tissue sample which has been stained with a conventional chemical stain using a machine-learning logic. Virtual-staining techniques bypasses the typically labor-intensive and costly histological staining procedures, and could be used as a blueprint for the virtual staining of tissue images acquired with other label-free imaging modalities. Virtual-staining approaches could be used for microguiding molecular analysis at the unstained-tissue level, by locally identifying regions of interest on the basis of virtual staining, and by using this information to guide subsequent analysis of the tissue, for example, microimmunohistochemistry or sequencing. This type of virtual microguidance on an unlabeled tissue sample might facilitate the high-throughput identification of disease subtypes and the development of customized therapies for patients.

Such prior art techniques face certain restrictions. In particular, the accuracy of the virtual stain may be limited. The flexibility in choosing different virtual stains may be limited.

To mitigate such limitations, WO 2021/198241 discloses a virtual stain determined based on multiple sets of imaging data depicting a tissue sample and having been acquired using multiple imaging modalities. The multiple imaging modalities are selected from the group comprising: hyperspectral microscopy imaging; fluorescence imaging; auto-fluorescence imaging; lightsheet imaging; digital phase contrast; and Raman spectroscopy.

While such techniques offer greater accuracy for the virtual stain, techniques such as hyperspectral microscopy imaging, fluorescence imaging, lightsheet imaging and Raman spectroscopy are relatively complex and time-consuming. Furthermore, the required light intensity and/or light dose may be significant, thereby posing a risk of damaging the sample.

### SUMMARY OF THE INVENTION

Accordingly, a need exists for advanced techniques of virtually staining a tissue sample. In particular, a need exists for determining an output image depicting the tissue sample having an accurate virtual stain. Robust prediction of the virtual stain is required.

A method of virtually staining of a tissue sample is disclosed. The method includes obtaining multiple sets of imaging data depicting a tissue sample. The multiple sets of imaging data have multiple different phase contrasts. The method also includes fusing and processing the multiple sets of imaging data in a machine-learning logic. The method further includes obtaining, from the machine-learning logic, at least one output image, each one of the at least one output image depicting the tissue sample comprising a respective virtual stain.

A computing device comprising at least one processor and a memory as disclosed. The at least one processor can load program code from the memory and execute the program code. Executing the program code causes the at least one processor to perform such method as disclosed above.

### BRIEF DESCRIPTION OF THE DRAWINGS

FIG. 1 schematically illustrates a workflow of virtually staining a tissue sample.

FIG. 2 schematically illustrates images having a virtual stain according to various examples.

FIG. 3 is a flowchart of a method according to various examples.

FIG. 4 illustrates a system for determining imaging data having a digital phase contrast.

FIG. 5 schematically illustrates a differential digital phase contrast according to various examples.

FIG. 6 schematically illustrates a transport of intensity digital phase contrast according to various examples.

FIG. 7 schematically illustrates the spatial frequency coverage of the differential digital phase contrast as well as of the transport of intensity digital phase contrast.

FIG. 8 illustrates a virtual stain according to examples.

FIG. 9 illustrates a virtual stain according to examples.

### DETAILED DESCRIPTION OF THE INVENTION

Some examples of the present disclosure generally provide for a plurality of circuits or other electrical devices. All references to the circuits and other electrical devices and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, machine-learning-specific hardware, e.g., a graphics processor unit (GPU) and/or a tensor processing unit (TPU), integrated circuits, memory devices (e.g. FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a set of program code that is embodied in a non-transitory computer readable medium programmed to perform any number of the functions as disclosed.

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are taken to be illustrative only.

The drawings are to be regarded as being schematic representations and elements illustrated in the drawings, which are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Hereinafter, techniques of imaging a sample are disclosed. When light interacts with a specimen of interest (sample), such as biological tissue, three primary contrast mechanisms may be used for image formation. First, the sample can attenuate the incident light due to absorption. Second, the sample can deform an incident optical wavefront, thereby imprinting phase contrast. Third, the light illuminating the sample may inelastically be scattered via fluorescence from either the sample itself (autofluorescence) or by means of chemical markers added to the sample.

For thin biological samples, for example tissue sections or adherent cell cultures, typically both absorption and autofluorescence effects are relatively weak. Therefore, biologists oftentimes resort to one of the two following light microscopy imaging modalities to assay structural information: (1) Phase contrast microscopes. This contrast modality allows for retaining the sample in its native state. Hardware and digital phase contrasts are known. (2) Chromogenic immunohistochemical stains or fluorescent markers may be used to chemically alter the contrast induced by the sample on the incident light. This contrast modality changes the chemical composition of the sample.

Various techniques are based on the finding that, among these two imaging modalities, phase contrast—in particular digital phase contrast—is relatively easy to attain, but it has been found that inferring chemically specific information from it is relatively challenging. Fluorescence markers can be used to label specific functional groups. However, both staining and fluorescence labeling have the disadvantage of requiring time-consuming sample preparation protocols and in some cases irreversibly changing the native state of the sample.

Recently, machine learning techniques, known as virtual staining or in-silico staining, have been reported, which allow for digitally transferring one image modality to another—an overview of the recent state of the art is found in Kreiss, Lucas, et al. “Digital staining in optical microscopy using deep learning—a review.” arXiv preprint arXiv:2303.08140 (2023).

Various techniques described herein generally relate to virtual staining of a tissue sample by utilizing a trained machine-learning logic (MLL). The MLL can be implemented, e.g., by a support vector machine or a deep neural network which includes at least one encoder branch and at least one decoder branch. Examples include a U-net, see Ronneberger, Olaf, Philipp Fischer, and Thomas Brox. “U-net: Convolutional networks for biomedical image segmentation.” International Conference on Medical image computing and computer-assisted intervention. Springer, Cham, 2015. The MLL be trained using a cyclic generative adversarial network, see e.g., Zhu, Jun-Yan, et al. “Unpaired image-to-image translation using cycle-consistent adversarial networks.” Proceedings of the IEEE international conference on computer vision. 2017. Such architecture includes a forward cycle and a backward cycle, each of the forward cycle and the backward cycle including a generator MLL and a discriminator MLL. Both the generator MLLs of the forward cycle and the backward cycle are respectively implemented using the MLL.

More specifically, according to various examples, multiple sets of imaging data can be fused and processed by the MLL. This is referred to as a multi-input scenario. An output image is provided. The output image depicts the tissue sample including a virtual stain, i.e., the output image can have a similar appearance as respective images depicting the tissue sample including a corresponding chemical stain. Thus, the virtual stain can have a correspondence in a chemical stain of a tissue sample stained using a staining laboratory process.

For example, the MLL can generate virtual H&E (Hematoxylin and Eosin) stained images of the tissue sample, and/or virtually stained images of the tissue sample highlighting HER2 (human epidermal growth factor receptor 2) proteins and/or ERBB2 (Erb-B2 Receptor Tyrosine Kinase 2) genes.

Another example would pertain to virtual fluorescence staining. For example, in life-science applications, images of cells—e.g., arranged ex-vivo in a multi-well plate—are acquired using transmitted-light microscopy. Also, a reflected light microscope may be used, e.g., in an endoscope or as a surgical microscope. It is then possible to selectively stain certain cell organelles, e.g., nucleus, ribosomes, the endoplasmic reticulum, the golgi apparatus, chloroplasts, or the mitochondria. A fluorophore (or fluorochrome, similarly to a chromophore) is a fluorescent chemical compound that can re-emit light upon light excitation. Fluorophores can be used to provide a fluorescence chemical stain. By using different fluorophores, different chemical stains can be achieved. For example, a Hoechst stain would be a fluorescent dye that can be used to stain DNA. Other fluorophores include 5-aminolevulinic acid (5-ALA), fluorescein, and Indocyanine green (ICG) that can even be used in-vivo. Fluorescence can be selectively excited by using light in respective wavelengths; the fluorophores then emit light at another wavelength. Respective fluorescence microscopes use respective light sources. It has been observed that illumination using light to excite fluorescence can harm the sample; this is avoided when providing virtual fluorescence staining. The virtual fluorescence staining mimics the fluorescence chemical staining, without exposing the tissue to respective excitation light.

According to examples, virtual staining is facilitated by multiple phase contrasts, e.g., hardware and/or digital phase contrasts. Various techniques disclosed herein to robustly transfer multiple sets of imaging data of a tissue sample having multiple different phase contrasts to other modalities, including fluorescence and immunohistochemical stains.

The choice between different phase contrasts is often dependent on the tissue sample under investigation and it can be beneficial to acquire multiple sets of imaging data with multiple phase contrast imaging modalities. Multiple phase contrasts are combined to robustify image transfer performance in virtual staining. More comprehensive information regarding the sample can be available to the MLL when using multiple phase contrasts. The virtual stain may be more accurately predicted. There may be a greater flexibility in predicting different types of virtual stain.

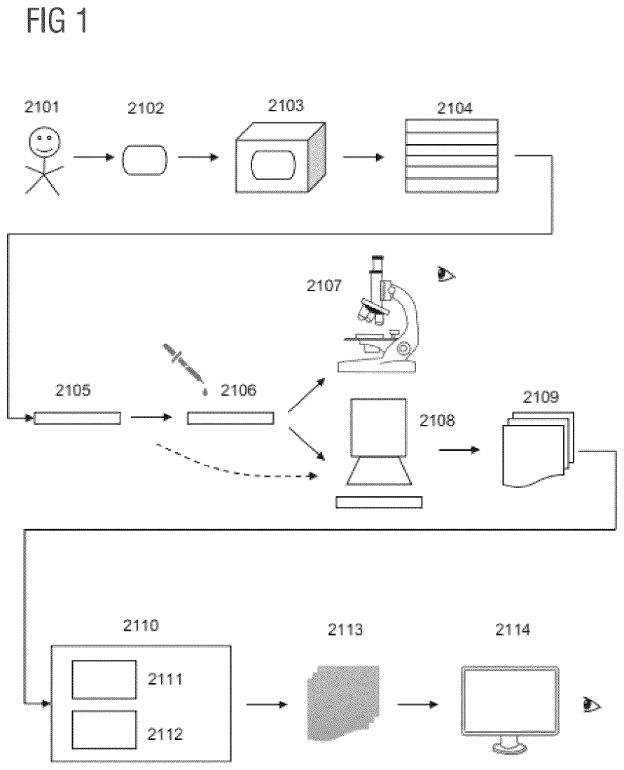

FIG. 1 illustrates aspects with respect to a workflow for generating images depicting a tissue sample including a stain, e.g., a chemical stain or a virtual stain. FIG. 1 schematically illustrates an example of a histopathology workflow. As explained above, virtual staining can also be applied in other use cases than histopathology. Then, different workflows for generating images can be applicable. For instance, for fluorescence imaging of cells, tissue samples including cell samples may be otherwise acquired and imaged in a respective microscope. Also, in-vivo imaging using an endoscope would be a possible use case for generating imaging data of tissue samples.

As shown in FIG. 1, for histopathology, tissue 2102 may be obtained from a living creature 2101 by surgery, biopsy or autopsy. After some processing steps to remove water and to prevent decay, said tissue 2102 may be embedded in a wax block 2103. From said block 2103, a plurality of slices 2104 may be obtained for further analysis. One slice of said plurality of slices 2104 may also be called a tissue sample 2105. Corresponding tissue samples 2015 may be obtained from adjacent slices.

As mentioned before, the tissue could also include cell samples or in-vivo inspection using, e.g., a surgical microscope or an endoscope.

Before analyzing the tissue sample 2105, a chemical stain may optionally be applied to the tissue sample 2105 using a staining laboratory process, to obtain a chemically-stained tissue sample 2106. In some examples, the tissue sample 2105 may also be directly analyzed (dashed arrow in FIG. 1). A chemically stained tissue sample 2106 may facilitate the analysis. In particular, chemical stains may reveal cellular components or generally well-defined structures of cells, which are difficult to observe in the unstained tissue sample 2105. Moreover, chemical stains may provide an increased contrast.

Applying a chemical stain may include a-priori transfecting or direct application of a fluorophore such as 5-ALA.

Traditionally, the tissue sample 2105 or 2106 is analyzed by an expert using a bright field microscope 2107.

Meanwhile, it has become more common to use image acquisition systems 2108 configured for acquiring digital image data of the tissue sample 2105 or the chemically stained tissue sample 2106 using one or more imaging modalities. Using different imaging modalities may facilitate acquiring imaging data 2109—e.g., 1-D, 2-D, or 3-D imaging data-of the tissue sample 2105.

The imaging data 2109 may be processed in a tissue analyzer 2110. The tissue analyzer 2110 may be implemented by a computer and/or by cloud processing at a server. The tissue analyzer 2110 may include a memory circuitry 2111 for storing the digital image data 2109 and/or program code, and may include a circuit 2112 for processing the digital image data 2109—e.g., upon loading the program code. The tissue analyzer 2110 may process the imaging data 2109 to provide one or more output images 2113 which may be displayed on a display 2114 to be analyzed by an examiner. For example, multiple output images 2113 depicting the tissue sample 2105, 2106 including different virtual stains may be provided. The tissue analyzer 2110 may comprise different types of trained or untrained machine-learning logic (details are with respect to the machine-learning logic are described below) for analyzing the non-stained tissue sample 2105 and/or the chemically stained tissue sample 2106 (i.e., the circuit 2112 can execute the machine-learning logic). The output images 2113 may depict the tissue sample 2105 with one or more virtual stains. The image acquisition system 2108 may be used for providing training data and/or reference images as a ground truth for training said machine-learning logic.

More generally, the tissue analyzer 2110 includes the circuit 2112 which may include a CPU and/or a GPU and/or a TPU. The circuit 2112 can load program code from the memory 2111. The circuit 2112 can execute the program code. Upon executing the program code, the circuit 2112 can perform one or more of the following logic operations as described throughout this disclosure: obtaining imaging data, e.g., via an input/output (I/O) interface of the tissue analyzer 2204 or by loading the imaging data from the memory; pre-processing the imaging data, e.g. to determine a digital phase contrast; virtual staining of the tissue sample depicted by the imaging data; executing a machine-learning logic to process the imaging data (inference); obtain at least one output image, from the machine-learning logic/when executing the machine-learning logic, e.g., to output the at least one output image via the I/O interface; setting parameters or hyper-parameters of the machine-learning logic when training the machine-learning logic; training the machine-learning logic, etc.

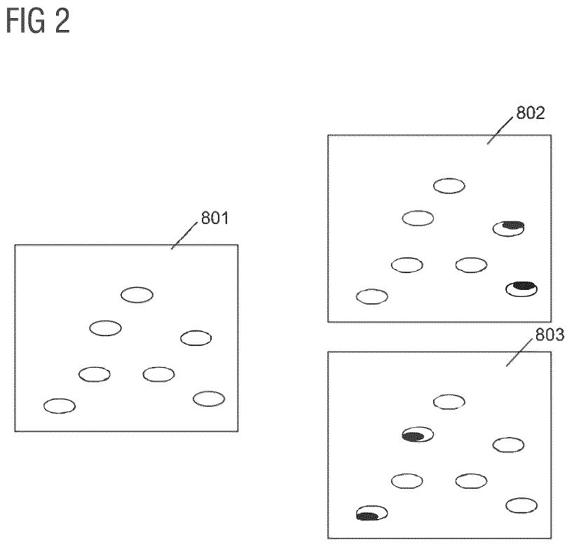

FIG. 2 schematically illustrates images 801-803 depicting a tissue sample. The image 801 depicts the tissue sample not including any chemical or virtual stain. For instance, the image 801 may have a digital phase contrast. Differently, the image 802 depicts the tissue sample including a chemical or virtual stain. Also, the image 803 depicts the tissue sample including a chemical virtual stain, wherein the chemical virtual stain of the tissue sample depicted by the image 803 is different from the chemical or virtual stain of the tissue sample depicted by the image 802: different structures or biomarker(s) are highlighted (full black areas in FIG. 2).

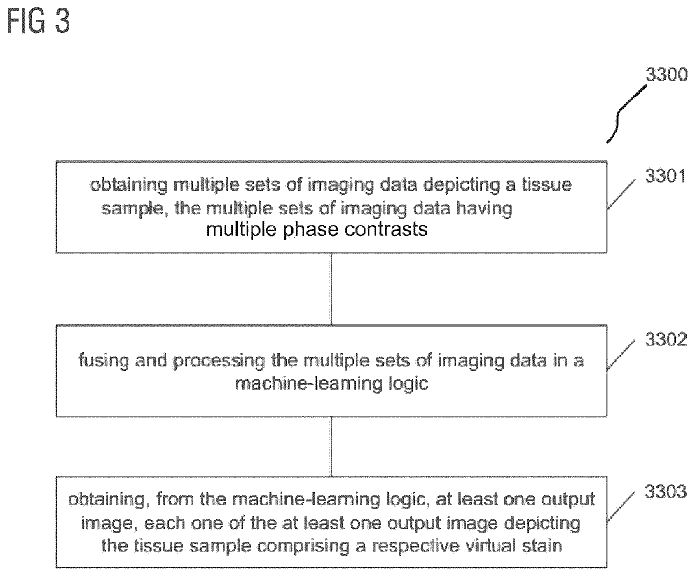

FIG. 3 is a flowchart of a method 3300 according to various examples. For example, the method 3300 according to FIG. 3 may be executed by at least one circuit—e.g., a CPU and/or a GPU and/or a TPU—upon loading program code from a nonvolatile memory. The method of FIG. 3 may be executed by the tissue analyzer 2110. The method of FIG. 3 facilitates virtual staining of a tissue sample.

At block 3301, multiple sets of imaging data depicting a tissue sample are obtained (e.g., loaded from a memory or obtained via an input interface from a data acquisition unit) and the multiple sets of imaging data have multiple phase contrasts. Each set of imaging data may include multiple instances of imaging data, e.g., multiple images (cf. FIG. 2: image 801) taken at different positions of the sample (e.g., for stitching) and/or at different times.

The multiple sets of imaging data may have different phase contrasts because they have been acquired using different phase-contrast imaging modalities. For instance, at least one of the multiple sets of imaging data may have been acquired using a digital phase contrast imaging modality. Examples include a differential digital phase contrast (DPC) and a transport of intensity (TIE) phase contrast. Other digital phase contrast imaging modalities include an inline holography phase contrast, an off-axis holography phase contrast, or a phase-shifting interferometry phase contrast. It would also be possible that at least one of the multiple sets of imaging data has been acquired using a non-digital (“classic” hardware-based) phase-contrast imaging modality. The multiple phase-contrast imaging modalities may include at least one of a Zernike phase contrast, a Jamin-Lebedeff interference phase contrast, and a shearing interferometry phase contrast.

It is not required in all scenarios that the multiple sets of imaging data having different phase contrasts have been acquired using different phase-contrast imaging modalities. For instance, at least two of the multiple sets of imaging data may have been acquired using the same phase-contrast imaging modality, but at different imaging settings. Examples would include a first set of the multiple sets of imaging data having been acquired using a given digital phase-contrast imaging modality such as DPC or TIE phase contrast, at a first wavelength (e.g., red or blue or yellow or infrared or ultraviolett) of the light; a second set of the multiple sets of imaging data having been acquired using that given digital phase-contrast imaging modality at a second wavelength of the light that is different than the first wavelength. Alternatively or additionally to using multiple wavelengths, it would also be possible to use multiple polarizations, e.g., left circular and right circular polarized light.

The tissue sample can be a cancer tissue sample removed from a patient, a tissue sample of other animals or plants.

The method 3300 of FIG. 3 optionally includes pre-processing after obtaining the multiple sets of imaging data, such as one or a combination of the following processing techniques: noise filtering; registration between imaging data of different sets of imaging data, for example, any pairs of the sets, etc.; resizing of the imaging data, etc.

Imaging data including one or more images having a digital phase contrast are also obtained from preprocessing raw images. For instance, for the DPC, multiple raw images of the tissue sample acquired at different angled illumination settings are combined. For instance, for the TIE, similarly multiple raw images of the tissue sample acquired at different defocus settings are combined. It would be possible that the method 3300 of FIG. 3 includes such preprocessing to obtain one or more sets of imaging data having multiple different phase contrasts.

At block 3302, the multiple sets of imaging data are fused and processed by an MLL. The MLL has been trained using supervised learning, semi-supervised learning, or unsupervised learning.

As a general rule, various implementations of the MLL are conceivable. In one example, a deep neural network may be used. For example, a U-net implementation is possible. See Ronneberger, Olaf, Philipp Fischer, and Thomas Brox. “U-net: Convolutional networks for biomedical image segmentation.” International Conference on Medical image computing and computer-assisted intervention. Springer, Cham, 2015.

More generally, the deep neural network can include multiple hidden layers. The deep neural network can include an input layer and an output layer. The hidden layers are arranged in between the input layer and the output layer. There can be a spatial contraction and a spatial expansion implemented by one or more encoder branches and one or more decoder branches, respectively. I.e., the x-y-resolution of respective representations of the imaging data and the output images may be decreased (increased) from layer to layer along the one or more encoder branches (decoder branches). At the same time, feature channels can increase and decrease along the one or more encoder branches and the one or more decoder branches, respectively. The one or more encoder branches and the one or more decoder branches are connected via a bottleneck. At the output layer or layers, the deep neural network can include decoder heads that include an activation function, e.g., a linear or non-linear activation function.

Thus, the MLL can include at least one encoder branch and at least one decoder branch. The at least one encoder branch provides a spatial contraction of respective representatives of the multiple sets of imaging data, and the at least one decoder branch provides a spatial expansion of the respective representatives of the at least one output image. It is, however, not required in all scenarios that the MLL implements spatial concentration and expansion. In other examples, the spatial resolution may not be affected (possibly with the exception of edge cropping).

For example, it would be possible that the MLL includes multiple encoder branches, one encoder branch for each of multiple sets of imaging data. Different encoder branches can be trained to process different phase contrasts.

As a general rule, the fusing of the multiple sets of imaging data may be implemented by concatenation or stacking of the respective representatives of the multiple sets of imaging data at at least one layer of the neural network. This may be an input layer (a scenario sometimes referred to as early fusion or input fusion) or a hidden layer (a scenario sometimes referred to as middle fusion or late fusion). For middle fusion, it would even be possible that the fusing is implemented at the bottleneck (sometimes referred to as bottleneck fusion). Where there are multiple encoder branches, the connection joining the multiple encoder branches defines the layer at which the fusing is implemented. As a general rule, it is possible that fusing of different pairs of imaging data is implemented at different positions, e.g., different layers.

Details with respect to processing multiple sets of imaging data having different contrasts are known from WO 2021/198241, the disclosure of which is incorporated herein by reference. Similar techniques can be used in the present disclosure for processing the multiple sets of imaging data having multiple different phase contrasts. Next, details with respect to digital phase contrasts will be explained.

At block 3303, at least one output image is obtained from the MLL 3500 and each one of the at least one output image depicts the tissue sample 3400 including a respective virtual stain. Some example virtual stains are: virtual H&E (Hematoxylin and Eosin) stained images of the tissue sample, virtually stained images of the tissue sample highlighting antibodies, such as anti-panCK, anti-CK18, anti-CK7, anti-TTF-1, anti-CK20/anti-CDX2, and anti-PSA/anti-PSMA, or other biomarkers. Further examples are primary IHC markers, e.g. HER2 (ERBB2), ER (Estrogen receptor/ESR1), PR (progesterone receptor/PGR); and proliferation markers, e.g. Ki-67 (MK167).

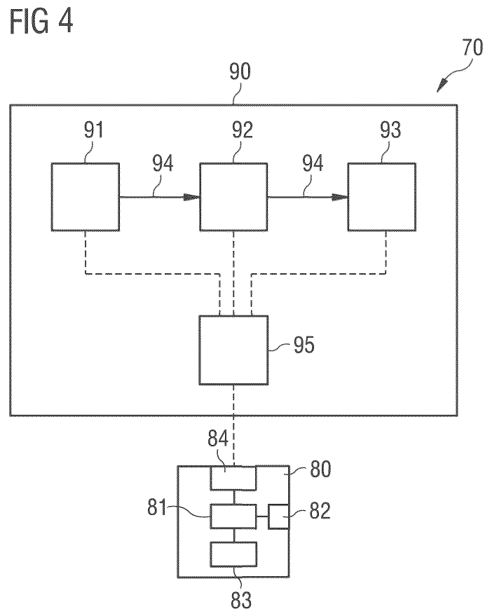

FIG. 4 schematically illustrates a system 70 for acquiring imaging data having a digital phase contrast. The system 70 includes a microscope 90 and a computer 80. The microscope 90 includes an illumination module 91, an optical system 92, and a detector module 93.

The system 70 can be a table-top optical microscope. The system 70 can be relatively compact and lightweight. This is an advantage of using a digital phase contrast such as TIE phase contrast or DPC.

The detector module 93 includes one or more cameras to acquire microscope images.

The illumination module 91 is configured to provide a switchable/re-configurable angled illumination of an imaging plane defined along the path of light 94 of the system 70. This means that the illumination angle can be controlled. Beyond controlling the main illumination angle, it would be optionally possible to also control the angular spectrum, e.g., the width or contributions, etc. For instance, it would be possible to activate multiple illumination configurations that have angular spectra of different width. Sometimes, only a single illumination direction may be activated (minimum width of the angular spectrum), sometimes multiple illumination directions may be superimposed (larger width of the angular spectrum). The illumination is partially coherent, e.g., using a point source, a collimated laser, or an areal light source. In practice, an array of Light Emitting Diodes (LED) is used, which exhibit a sufficiently high degree of spatial and temporal coherence.

The optical system 92 is configured to illuminate the imaging plane and further image the imaging plane onto the at least one camera of the detector module 93.

The microscope 90 also includes a control module 95 that is configured to control the various components of the microscope. For example, the control module 95 may be implemented using a CPU or a Field Programmable Gated Array (FPGA) or an Application Specific Integrated Circuit (ASIC). The control module 95 can include memory. The control module 95 can be configured to control the illumination module to activate multiple angled illumination configurations. The control module 95 can also be configured to control the detector module 93 and, specifically, the at least one camera of the detector module 93 to acquire multiple images. Optionally, the control module 95 can be configured to control the optical system 92, e.g., by moving a sample holder configured to hold a sample, or by driving a movable nosepiece holding an objective, or more generally implementing multiple defocus values.

Also illustrated is the computer 80 that includes an interface 84 configured to communicate with the microscope 90, specifically the control module 95. For instance, a processor 81 of the computer 80—e.g., a CPU, an FPGA or an ASIC—can provide control data to the control module 95 to implement certain control functionality, e.g., trigger image acquisition, trigger activation of certain angled illumination configurations, trigger implementation of certain defocus values, etc. The processor 81 can also retrieve image data from the microscope 90 via the interface 84 and post process the image data. The processor 81 can provide sets of imaging data having multiple different digital phase contrasts to the tissue analyzer 2110.

The processor 81 is configured to load program code from a memory 83 and execute the program code to perform such techniques. In particular, digital postprocessing for determining a phase contrast image based on multiple intensity images retrieved from the microscope 90 can be executed by the processor 81. It is possible that the computer 80 also includes a user interface 82, e.g., a GUI, to output phase contrast images thus determined.

The computer 80 may implement the tissue analyzer 2110. Thereby, edge-inference of the MLL becomes possible for providing a virtual stain.

While in FIG. 4 a scenario is illustrated in which the computer architecture split in between the control module 95 and the computer 80, in some scenarios it would also be possible that digital postprocessing of image data is executed by the control module 95. Alternatively or additionally, component level control of the various components of the microscope 90 can also be a task at least partly delegated to the computer 80.

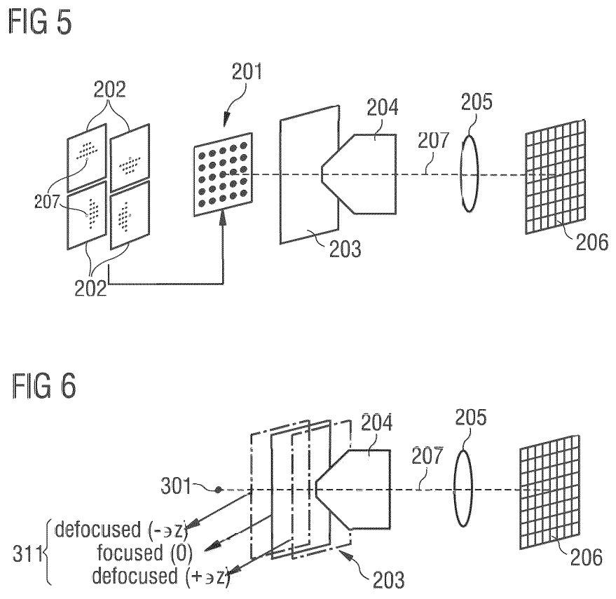

FIG. 5 and FIG. 6 illustrate the experimental hardware utilized in the acquisition of a differential phase contrast (DPC) phase contrast and a TIE phase contrast, respectively.

DPC (FIG. 5) uses a programmable illumination unit (PIU) 201. It requires an acquisition of at least three focal-plane images under varying illumination patterns 202 to produce a phase contrast image of a sample. In particular, these variable illumination patterns 202 correspond to different angled illumination configurations for illuminating the imaging plane 203 at different angles while the sample is kept in a fixed position. Each pattern 202 includes activated LEDs at different asymmetric distributions with respect to the optical axis 207 (dashed line and open circle). I.e., the respective illumination configuration includes multiple illumination directions (defined by the activated LEDs). An objective 204 and a tube lens 205 are used (part of the optical system, cf. FIG. 1: optical system 92) to image the imaging plane 203 onto a pixelated detector 206 (camera). Different LEDs are activated for the different variable illumination patterns 202. A subsequent numerical deconvolution routine converts the recorded images into a phase contrast image of the sample, see Tian, Lei, and Laura Waller. “Quantitative differential phase contrast imaging in an LED array microscope.” Optics express 23.9 (2015): 11394-11403.

Now referring to FIG. 6: TIE phase contrast is based on a diffusion equation which relates an axial intensity derivative to the phase of the sample. The axial intensity derivative can be approximated by recording at least two images, where the imaging plane and the sample is shifted axially and close to the focal plane, thereby implementing multiple defocus values 311. The illumination unit is ideally fully coherent, such as an on-axis point source 301 or an on-axis collimated laser. After recording images under defocus variation (also known as z-stack), sufficient information is available to solve the underlying diffusion equation to yield the phase information of the sample, see Streibl, Norbert. “Phase imaging by the transport equation of intensity.” Optics communications 49.1 (1984): 6-10. This includes a numerical deconvolution routine that converts the recorded images into a phase contrast image of the sample. The TIE is a diffusion equation which relates an axial intensity derivative to the phase of the sample. The axial intensity derivative can be approximated by recording at least two images, where the sample is shifted axially and close to the focal plane.

Various techniques are based on the finding that particular beneficial input data for the MLL can be obtained by combining, firstly, DPC with, secondly, TIE phase contrast. This is because the spatial frequencies within the numerical aperture covered by DPC on the one hand and TIE on the other hand are complementary. Thus, by fusing, in the MLL, the first set of imaging data having the DPC contrast with the second set of imaging data having the TIE phase contrast, more comprehensive information is available for the input to the MLL. Since both DPC and TIE phase contrast are digital phase contrasts using similar acquisition hardware—as explained above—the acquisition process for acquiring the multiple sets of imaging data can be implemented using one and the same hardware. More specifically, a table-top optical microscope (cf. FIG. 4) may be sufficient for acquiring high-quality sets of imaging data having multiple phase contrasts. This has the advantage of enabling edge-inference of the MLL at a compute circuitry co-deployed at a user site with the table-top optical microscope. The exposure of the sample to light is also limited, e.g., if compared to fluorescence or interferometric techniques.

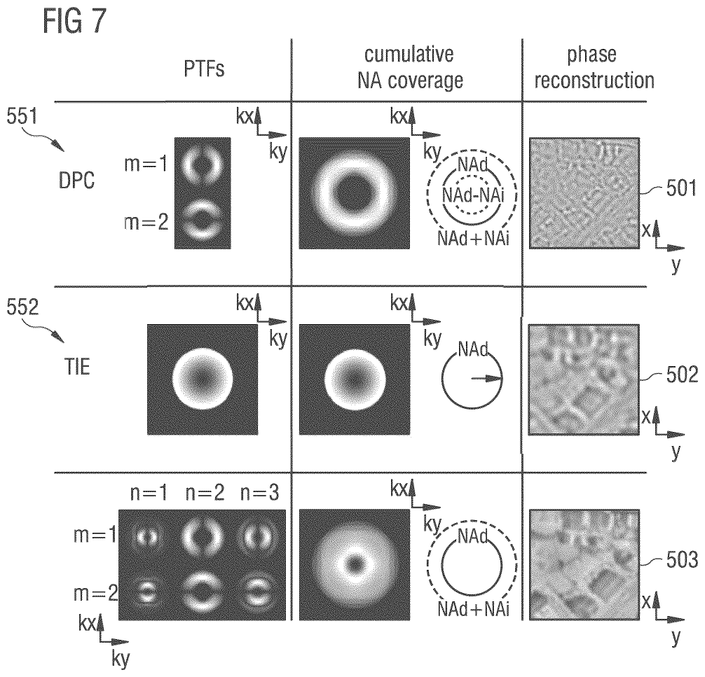

FIG. 7 illustrates the PTFs for different digital phase contrast techniques—DPC, TIE, and a combination thereof—and the associated achievable numerical aperture (NA) coverage. An example phase image 501-503 is shown for each technique.

FIG. 7 illustrates the process of tailoring the spatial frequency coverage of d(k). In what follows the quantity \sqrt{d(k)} is referred to as cumulative NA coverage.

FIG. 7 first row illustrates DPC 551 (cf. FIG. 5) under unmatched illumination condition (numerical aperture of the illumination part of the optical system NAi=0.3, and numerical aperture of the detector part of the optical system NAd=0.6). While DPC is principally capable of reaching a cumulative NA radius of NAi+NAd (radius of the dashed line in the middle of the first row of FIG. 7), the center of the NA coverage exhibits a hole approximately of radius NAd−NAi, resulting in a loss of low spatial frequency coverage. This in turn causes the resulting phase reconstruction to exhibit low contrast (as apparent from the phase contrast image 501).

TIE phase contrast 552—shown in FIG. 7, second row (cf. FIG. 6)—employs axial defocusing. When evaluating the corresponding phase transfer function (FIG. 7, second row, left column), it is seen that the NA coverage reaches lower values in k-space as compared to DPC (simply speaking, the black circle around the k-space center is smaller). This results in an improved contrast in the final phase reconstruction as compared to DPC (cf. phase contrast image 502). However, because only a single on-axis point source is used, standard TIE phase contrast systems have a vanishing illumination NA. Thus, the NA coverage reaches only a bandwidth of NAd (simply speaking, the white donut has a limited radius), which is a disadvantage as compared to DPC in terms of achievable lateral resolution (which is given by λ/[NAᵢ+NA_{d}], where λ is the wavelength).

Next, FIG. 7, third row illustrates the spatial frequency coverage available to the virtual staining MLL if two sets of imaging data having, firstly, a DPC and, secondly, a TIE phase contrast are combined. By fusing the PTFs from both variable illumination patterns and defocus, a wider range of spatial frequencies can be covered (the donut in the middle column has a smaller inner hole and a wider radius). The resulting phase reconstruction (right column) features superior phase contrast as compared to DPC, while reaching a higher resolution as compared to TIE phase contrast (cf. phase contrast image 503).

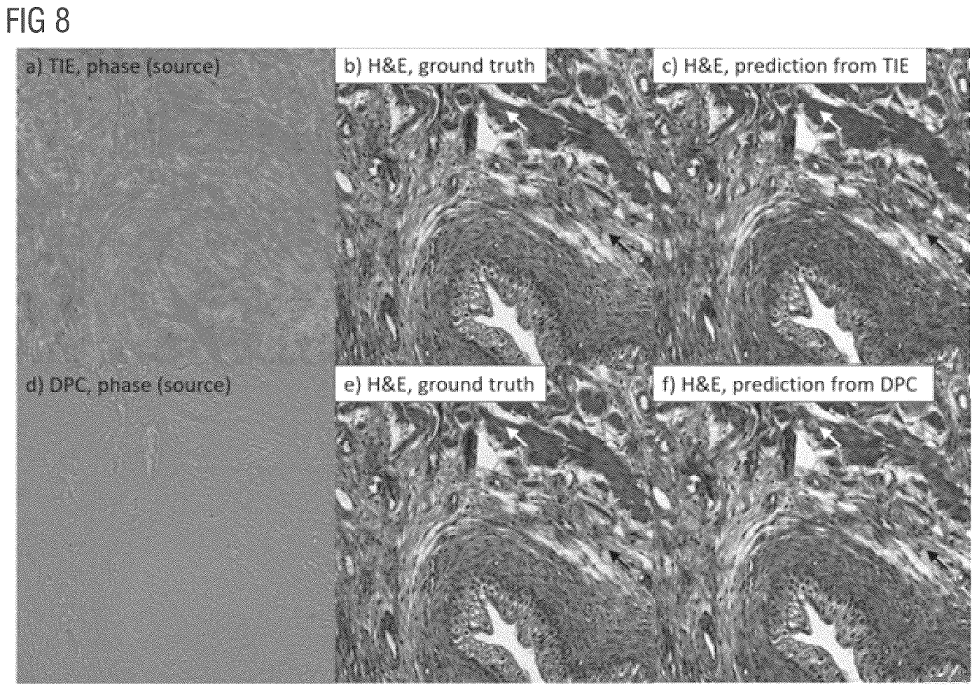

FIG. 8 shows a virtual staining result where a U-net was trained to transfer phase contrast images to H&E stained images. A first imaging data set is based on the TIE digital phase contrast and a second imaging data set is based on the DPC phase contrast, both using only a red LED. In addition red, green and blue bright field images of the same sample were acquired. This procedure was repeated for various different regions of interest. The acquired data was then used to train the U-net. Panel a) shows a phase contrast image that was obtained from a defocused stack via solving the transport of intensity equation (TIE phase contrast). This image contains visible artefacts, as witnessed by the local inhomogeneities in the displayed gray values. The reduced quality of this TIE phase contrast image (as compared to the DPC in panel d) is caused by a violation of the non-absorbing specimen assumption inherent in TIE. Nevertheless, the H&E prediction from the TIE phase contrast image in panel c) bears a high resemblance to the ground truth data shown in panel b), However, closer inspection reveals certain shortcomings. The black arrow points to a region where fine details cannot be predicted reliably by the TIE phase contrast image (only two out of three black dots are visible). In panel d), the DPC phase contrast image is shown. This DPC phase contrast image was used to predict the H&E image, shown in panel f. Again, this image bears close resemblance to the ground truth (reproduced in panel e) to facilitate comparison). The black arrow shows the same region as previously in panel c), but now the three black dots were correctly predicted, as judged by comparison with the ground truth. The black arrows thus highlight regions where the DPC phase contrast image produces superior predictions as compared to the TIE phase contrast image. Conversely, also regions are identified where the TIE phase contrast image produced superior H&E predictions as compared to the DPC phase contrast image. This is the case for the red tissue region indicated by the white arrow. Here the ground truth and the prediction from the TIE phase image display a homogeneous red region (compare white arrows in panels b and c), while the H&E prediction from DPC incorrectly predicts an inhomogeneous tissue region (compare panels e and f).

The example illustrated in FIG. 8 suggests that DPC and TIE phase contrast images have different prediction performance for virtual staining applications. Next, an MLL fusing both digital phase contrast imaging modalities has been assessed.

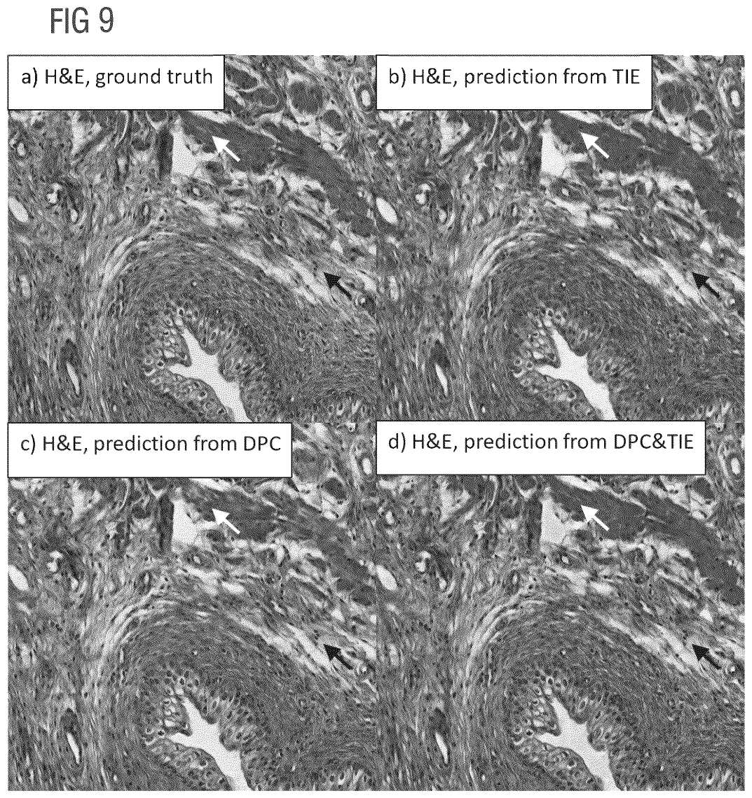

FIG. 9 shows the results for training a virtual staining neural network with both DPC and TIE phase contrast source images. Comparing again the black arrows, which indicate the ability to predict detailed structures, and the white arrows, which indicate the ability to predict homogeneous background, one sees that that providing both DPC and TIE phase contrast images (panel d) as input for virtual staining MLL outperforms using only a single source image (either TIE in panel b or DPC in panel c but not both).

In summary, multimodal phase contrast images are provided as input for virtual staining. This improves the robustness of the predicted images. Above, primarily a combination of a first set of imaging data having DPC and a second set of imaging data having TIE phase contrast have disclosed; however, various other combinations of sets of imaging data having phase contrast are possible. Possible examples are disclosed next: Multiple sets of imaging data having classical phase contrast can be combined, e.g., including Zernike phase contrast, differential interference contrast (DIC), and Jamin-Lebedeff interference contrast. Alternatively or additionally, alternative digital phase contrast methods can be used, beyond the DPC and TIE phase contrast. Examples including shearing interferometry, inline and off-axis holography, phase-shifting interferometry. Alternatively or additionally to using different phase contrast imaging modalities, a single phase contrast imaging modality can be used under variation of other optical parameters. A first example pertains to variation of color/wavelength. For example, a plurality of colors can be used in a single phase contrast experimental setup, for example DPC employing an LED array with red, green, and/or blue illumination channels (and similar for the aforementioned phase contrast methods). Likewise, multiple spectral bands in the infrared or UV can be utilized as input to robustify predictions by the MLL. A second example pertains to a variation of polarization. This includes varying the principal axis or linear and/or circular polarization of the light either upstream and/or downstream of the sample.

Although the invention has been shown and described with respect to certain preferred embodiments, equivalents and modifications will occur to others skilled in the art upon the reading and understanding of the specification. The present invention includes all such equivalents and modifications and is limited only by the scope of the appended claims.

What is claimed is:

1. A method of virtually staining a tissue sample, the method comprising:
obtaining multiple sets of imaging data depicting a tissue sample, the multiple sets of imaging data having multiple different phase contrasts,
fusing and processing the multiple sets of imaging data in a machine-learning logic, and
obtaining, from the machine-learning logic, at least one output image, each one of the at least one output image depicting the tissue sample comprising a respective virtual stain.

2. The method of claim 1,
wherein the multiple different phase contrasts comprise at least two different phase contrasts that have been acquired using multiple different phase-contrast imaging modalities.

3. The method of claim 2,
wherein the multiple different phase-contrast imaging modalities comprise a first digital phase-contrast imaging modality and a second digital phase-contrast imaging modality,
wherein the first digital phase-contrast imaging modality is a transport of intensity equation digital phase-contrast imaging modality, and
wherein the second digital phase-contrast imaging modality is a differential digital phase-contrast imaging modality.

4. The method of claim 2,
wherein the multiple different phase-contrast imaging modalities comprise at least one of a Zernike phase contrast, a Jamin-Lebedeff interference phase contrast, a shearing interferometry phase contrast, an inline holography phase contrast, an off-axis holography phase contrast, or a phase-shifting interferometry phase contrast.

5. The method of claim 1,
wherein the multiple different phase contrasts comprise at least two different phase contrasts that have been acquired using the same phase-contrast imaging modality at different imaging settings.

6. The method of claim 5,
wherein the at least two different phase contrasts have been acquired using a digital phase-contrast imaging modality at multiple different wavelengths.

7. The method of claim 5,
wherein the at least two different phase contrasts have been acquired using a digital phase-contrast imaging modality at multiple different polarizations.

8. A computing device comprising at least one processor and a memory, wherein the at least one processor is configured to load program code from the memory and to execute the program code, wherein the at least one processor, upon executing the program code, performs the following steps:
obtaining multiple sets of imaging data depicting a tissue sample, the multiple sets of imaging data having multiple different phase contrasts,
fusing and processing the multiple sets of imaging data in a machine-learning logic, and
obtaining, from the machine-learning logic, at least one output image, each one of the at least one output image depicting the tissue sample comprising a respective virtual stain.

9. The computing device of claim 8,
wherein the computing device is coupled with an optical imaging system configured to acquire the multiple sets of imaging data, and
wherein the processor is configured to load the multiple sets of imaging data from the optical imaging device.

10. The computing device of claim 8,
wherein the at least one processor is configured to execute the method of claim 1.

11. The computing device of claim 9,
wherein the at least one processor is configured to execute the method of claim 1.
